(19) United States
(12) Patent Application Publication
(10) Pub. No.: US 2023/0034832 A1
(43) Pub. Date: Feb. 2, 2023

(54) Protective Drape For a Medical Procedure and Method of Use

(71) Applicant: Typenex Medical, LLC, Chicago, IL (US)

(72) Inventor: Christopher Walters, Duxbury, MA (US)

(21) Appl. No.: 17/875,089

(22) Filed: Jul. 27, 2022

Related U.S. Application Data
(60) Provisional application No. 63/203,654, filed on Jul. 27, 2021.

(51) Int. Cl. A61B 46/20; A61B 46/10

(52) U.S. Cl. CPC A61B 46/20; A61B 46/10; A61M 2025/0266

(57) ABSTRACT

A protective drape for a medical procedure may include a protective sheet including an interior surface, an exterior surface, a first end, a second end, a first side, and a second side. A first flap is coupled to the interior surface of the protective sheet so as to form a pocket at the first end of the protective sheet. The first flap includes an outer edge positioned away from the second end of the protective sheet and connected to the protective sheet, and a first lateral edge that is connected to the protective sheet so as to form a sealed periphery of the pocket that extends over the outer edge and a first lateral edge of the first flap.

### CROSS-REFERENCE TO RELATED APPLICATION

The present application claims priority to U.S. Provisional Application No. 63/203,654, filed Jul. 27, 2021, which is hereby incorporated by reference.

### BACKGROUND

During medical procedures, a patient may expel fluids by coughing, breathing, sweating or other means. Likewise, devices and instruments that contact the patient may also spread contaminants from the patient. Containment of such fluids can help maintain a clean environment and protect medical personnel.

### SUMMARY

In one aspect, a protective drape for a medical procedure is disclosed. An example protective drape may include a protective sheet including an interior surface, an exterior surface, a first end, a second end, a first side, and a second side. A first flap may be coupled to the interior surface of the protective sheet so as to form a pocket at the first end of the protective sheet. The first flap may include an outer edge positioned away from the second end of the protective sheet and connected to the protective sheet, and a first lateral edge that is connected to the protective sheet so as to form a sealed periphery of the pocket that extends over the outer edge and a first lateral edge of the first flap.

In an aspect, a protective drape is disclosed, where the first flap may include a second lateral edge that is also connected to the protective sheet so as to continue the sealed periphery of the pocket over the second lateral edge of the first flap.

In an aspect, a protective drape is disclosed, where the first flap may extend across the protective sheet from the first side to the second side.

In an aspect, a protective drape is disclosed, where the first end of the protective sheet may include a first edge, and where the first edge of the protective sheet may be aligned with the outer edge of the first flap.

In an aspect, a protective drape is disclosed, where the first flap and the protective sheet may be formed by a single material sheet, and where the first edge of the protective sheet and the outer edge of the first flap may be connected by a fold in the single material sheet.

In an aspect, a protective drape is disclosed, where the first lateral edge of the first flap may be connected to the protective sheet by a seam.

In an aspect, a protective drape is disclosed, where the protective drape may further include a first wing extending laterally outward from the first side of the protective sheet.

In an aspect, a protective drape is disclosed, where the protective sheet and the first wing are formed by a single material sheet.

In an aspect, a protective drape is disclosed, where the protective drape may further include an aperture extending through a central region of the protective sheet.

In an aspect, a protective drape is disclosed, where the protective drape may further include a second flap coupled to the interior surface of the protective sheet so as to form a second pocket at the second end of the protective sheet. The second flap may include an outer edge positioned away from the first end of the protective sheet and connected to the protective sheet, and a first lateral edge that is connected to the protective sheet so as to form a sealed periphery of the pocket that extends over the outer edge and a first lateral edge of the second flap.

In an aspect, a protective drape is disclosed, where the protective drape may further include an adhesive strip secured to the protective sheet adjacent to the aperture, the adhesive strip being configured to attach to a tube passing through the aperture.

In an aspect, a protective drape is disclosed, where the adhesive strip may be attached to the exterior surface of the protective sheet.

In an aspect, a protective drape is disclosed, where the protective drape may further include an adhesive strip secured to the protective sheet at the second end of the protective sheet, the adhesive strip being configured to attach to a tube passing adapted for use with the protective drape.

In an aspect, a protective drape is disclosed, where the protective sheet may be transparent.

In an aspect, a protective drape is disclosed, where the protective sheet may be formed of a polyfilm material.

In another aspect, a method is disclosed. An example method may include removing a distal end of an airway tube from a patient, where a proximal end of the airway tube is secured to a protective drape according to the disclosure. The method may further include securing the distal end of the airway tube over the interior surface of the protective drape such that fluids from the airway tube drip onto the protective drape and run into the pocket.

In accordance with this disclosure, the protective drapes described herein encompass devices that in other contexts may be referred to as bibs, covers, or barriers.

### BRIEF DESCRIPTION OF THE DRAWINGS

The accompanying drawings, which are included to provide a further understanding of the disclosure, are incorporated in and constitute a part of this specification, illustrate embodiments of the disclosure, and together with the detailed description serve to explain the principles of the invention. No attempt is made to show structural details of the invention in more detail than may be necessary for a fundamental understanding of the invention and various ways in which it may be practiced.

FIG. 1 shows a protective drape, according to an example embodiment;

FIG. 2 shows a patient that is intubated with an airway tube and the protective drape of FIG. 1 covering the patient;

FIG. 3 shows the patient of FIG. 2 with an anesthesia circuit disconnected from the airway tube;

FIG. 4 shows the protective drape and airway tube of FIG. 2 after removal from the patient;

FIG. 5 shows a protective drape, according another example embodiment;

FIG. 6 shows a protective drape according to another example embodiment; and

FIG. 7 is a flow chart of a method according to an example embodiment.

### DETAILED DESCRIPTION

### I. Introduction

Disclosed herein are protective drapes and methods of using protective drapes. In an example, such a protective drape may be used in a medical procedure to cover an intubated patient. The protective drape can help contain liquids, such as airborne droplets or projected liquids, that are expelled from the patient. This liquid containment may enhance the safety of medical personnel and limit the spread of contaminants. Moreover, the protective drape may help limit the spread of contaminants from equipment used during an extubation process, such as an airway tube that is removed from the patient, and may be used to contain the airway tube or other equipment after it is removed from the patient.

### II. Example Apparatus

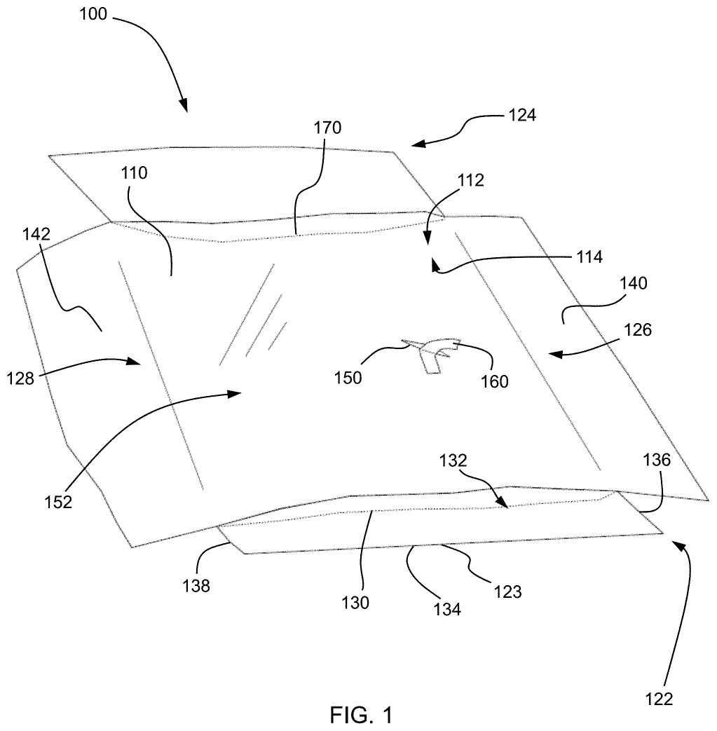

FIGS. 1-6 show protective drapes and aspects of protective drapes, according to example embodiments. FIGS. 1-6 are provided for purposes of illustration only and components of apparatus depicted in the Figures are not to scale. Further, components of apparatus depicted in the Figures with the same or similar reference numerals in different Figures may take the same or similar form and operate in the same or similar manner unless otherwise noted.

FIG. 1 shows protective drape 100 according to an example embodiment. The protective drape may include a protective sheet 110 including an interior surface 112, an exterior surface 114, a first end 122, a second end 124, a first side 126, and a second side 128. A first flap 130 may be coupled to the interior surface 112 of the protective sheet 110 so as to form a pocket 132 at the first end 122 of the protective sheet 110. The first flap 130 may include an outer edge 134 positioned away from the second end 124 of the protective sheet 110 and a first lateral edge 136. The outer edge 134 and first lateral edge 136 may each be connected to the protective sheet 110 so as to form a sealed periphery of the pocket 132 that extends over the outer edge 134 and the first lateral edge 136 of the first flap 130.

In some embodiments, the first flap 130 may include a second lateral edge 138 that is also connected to the protective sheet 110 so as to continue the sealed periphery of the pocket 132 over the second lateral edge 138 of the first flap 130. For example, in FIG. 1, the first flap 130 has a rectangular shape with both a first lateral edge 136 and a second lateral edge 138 that form the pocket 132. In other embodiments, the first flap 130 may have a triangular shape and include an outer edge 134 and a first lateral edge 136 without a second lateral edge. Other shapes of the first flap that forms the pocket are also possible.

In some embodiments, the first flap 130 may extend across the protective sheet 110 from the first side 126 to the second side 128. Accordingly, the pocket 132 may catch any fluids traveling over any portion across the width of the protective sheet 110. In other embodiments, the first flap 130 may extend over only a portion of the protective sheet. For example, in some embodiments, the first flap 130 and the pocket 132 formed by the first flap 130 may be positioned only in one corner of the protective sheet 110.

In some embodiments, the first end 122 of the protective sheet 110 may include a first edge 123. The first edge 123 of the protective sheet 110 may be aligned with the outer edge 134 of the first flap 130. In other embodiments, the protective sheet 110 may extend past the pocket 132 formed by the first flap 130. In other words, in some embodiments, the pocket 132 may be positioned inward from the outer edge formed at the first end 122 of the protective sheet.

In some embodiments, the first flap 130 and the protective sheet 110 may be formed by a single material sheet. For example, the first edge 123 of the protective sheet 110 and the outer edge 134 of the first flap 130 may be connected by a fold in the single material sheet. In other embodiments, the first lateral edge 136 of the first flap 130 and a lateral edge of the protective sheet 110 may be formed by a fold in the single material sheet. Further still, in some embodiments, the first flap 130 and protective sheet 110 may be formed from separate sheets that are attached to one another.

In some embodiments, the first lateral edge 136 of the first flap 130 may be connected to the protective sheet 110 by a seam. Further, in some embodiments, the outer edge 134 of the first flap 130 may be connected to the protective sheet 110 by a seam. Moreover, in some embodiments, both the first lateral edge 136 and the outer edge 134 of the first flap 130 may be connected by seams.

In some embodiments, the protective drape may further include a first wing 140 extending laterally outward from the first side 126 of the protective sheet 110. Further, in some embodiments, the protective drape may include a second wing 142 extending laterally outward from the second side 128 of the protective sheet 110. Moreover, in some embodiments the protective drape 100 may include wings on both sides of the protective sheet, while in other embodiments, the protective drape 100 may include a wing on only one side. It should be understood that the first side of the protective sheet may be the left side or the right side, and thus a protective drape with a single wing connected to the first side of the protective sheet may be on the left side or the right side of the protective drape.

In some embodiments, the protective sheet 110 and the first wing 140 may be formed by a single material sheet. In other embodiments, the first wing 140 may be connected to the protective sheet 110 by a seam.

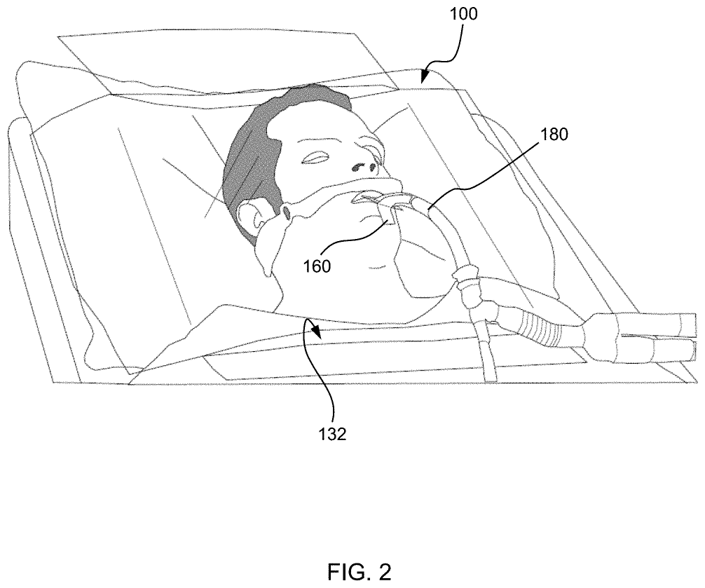

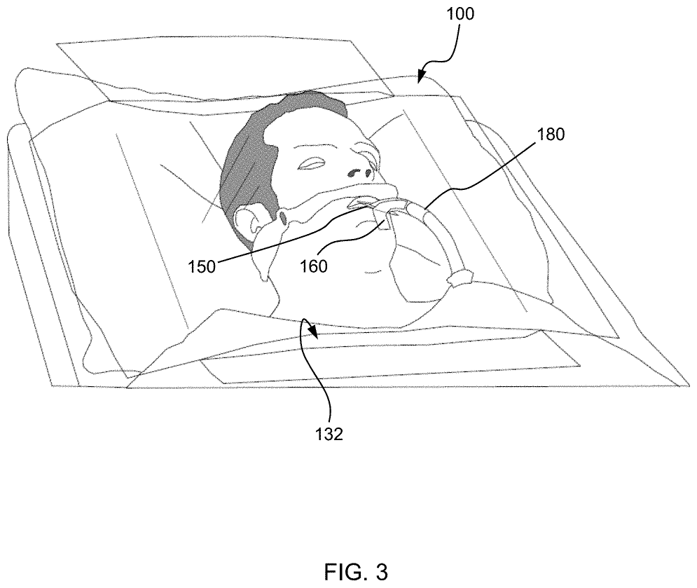

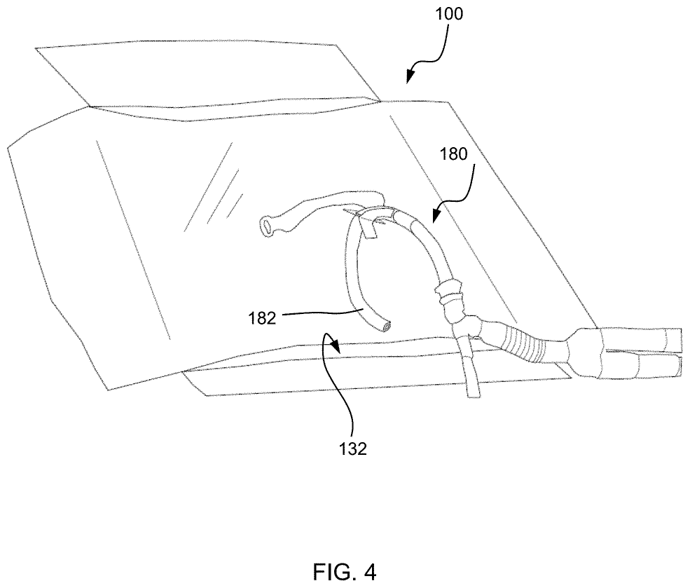

In some embodiments, the protective drape 100 may further include an aperture 150 extending through a central region 152 of the protective sheet 110. For example, the aperture may be used to extend a medical component, such as an airway tube, through the protective drape 100. For example, FIG. 2 shows an embodiment of a protective drape 100 in use in a medical procedure. The protective drape 100 shown in FIG. 2 is covering a patient and an airway tube 180 is extending through the aperture 150 in the protective sheet 110 and into the patient's mouth. FIG. 2 shows the patient intubated with an anesthesia circuit connected to the airway tube 180. FIG. 3 shows the same patient with the anesthesia circuit disconnected from the airway tube. Further, FIG. 4 shows the airway tube 180 after removal from the patient, where the distal end 182 of the airway tube is visible. After the airway tube is removed from the patient it may be wrapped in the protective drape 100 and positioned so that fluids are captured in the pocket 132, thereby containing potentially hazardous fluids from the patient.

In some embodiments, the protective drape 100 may further include a second flap 170 coupled to the interior surface 112 of the protective sheet 110 so as to form a second pocket 172 at the second end 124 of the protective sheet 110. The second flap 170 may include an outer edge 174 positioned away from the first end 122 of the protective sheet 110 and connected to the protective sheet 110. Second flap 170 may also include a first lateral edge 176 that is connected to the protective sheet 110 so as to form a sealed periphery of the pocket 172 that extends over the outer edge 174 and a first lateral edge 176 of the second flap 170.

In some embodiments, the protective drape 100 may further include an adhesive strip 160 secured to the protective sheet 110 adjacent to the aperture 150. The adhesive strip 160 may be configured to attach to a tube passing through the aperture 150. For example, as shown in FIG. 2, the adhesive strip 160 is secured to the airway tube 180 that is inserted into the intubated patient's mouth.

In some embodiments, the adhesive strip 160 may be attached to the exterior surface 114 of the protective sheet 110. In other embodiments, the adhesive strip 160 may be attached to the interior surface 112 of the protective sheet 110. Still, in other embodiments, the protective drape 100 may be secured to an airway tube by another method.

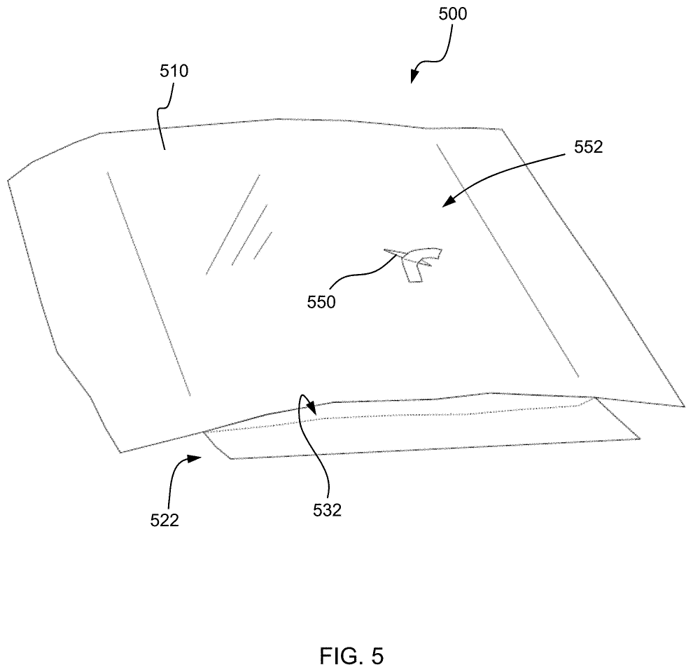

FIG. 5 shows another example embodiment of a protective drape 500 that includes a single pocket 532 disposed at the first end 522. The protective drape 500 includes an aperture 550 disposed in a central region 552 of the protective sheet 510, and is otherwise similar to protective drape 100, except that protective drape 500 does not include a second flap and pocket.

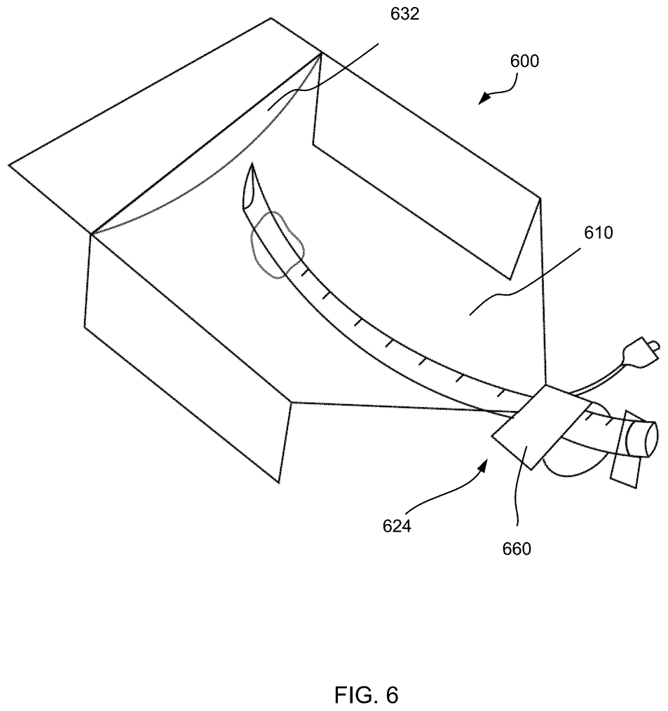

FIG. 6 shows another example embodiment of a protective drape 600 that also includes a single pocket 632. In the embodiment illustrated in FIG. 6, an adhesive strip 660 may be secured to the protective sheet 610 at the second end 624 of the protective sheet 610. The adhesive strip 660 may be configured to attach to a tube adapted so as to hold the protective drape 600 against such a tube. Accordingly, the protective drape 600 may be operable with an airway tube without including an aperture through the drape.

In some embodiments, the protective sheet may be transparent. Further, in some embodiments, the protective sheet may be formed of a polyfilm material. For example, in some embodiments, the protective sheet may be formed of HDPE, LDPE, LLDPE, or another polyfilm material. Likewise, in some embodiments, the first flap may also be formed of a polyfilm material. Further, in some embodiments, additional flaps or wings may also be formed of a polyfilm material. Moreover, in some embodiments, the protective sheet and any flaps and wings may be formed of the same material.

### III. Example Method

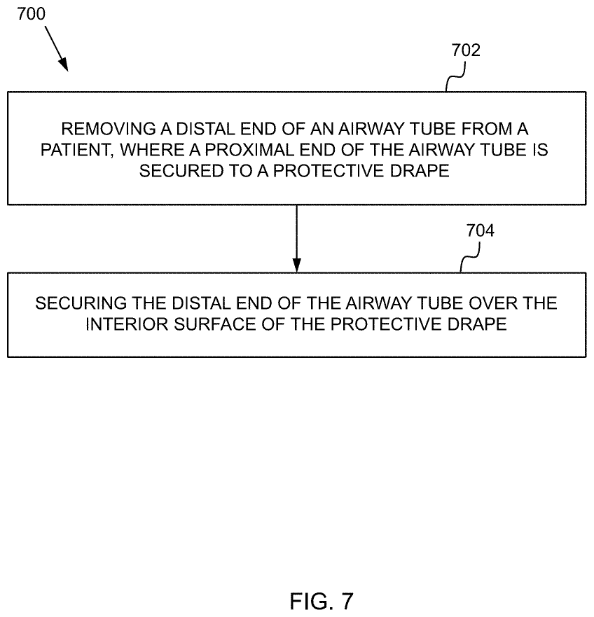

FIG. 7 shows an example embodiment of a method 700 for a medical procedure. In some embodiments, the method 700 may be performed in connection with protective drape according to any of the embodiments described herein. In some embodiments, the method 700 may be performed in connection with extubation of a patient.

As shown by block 702, the method 700 may involve removing a distal end of an airway tube from a patient, wherein a proximal end of the airway tube is secured to a protective drape. Further, the method 700 may involve, as shown by block 704, securing the distal end of the airway tube over the interior surface of the protective drape such that fluids from the airway tube drip onto the protective drape and run into the pocket.

In some embodiments, the method may involve securing the protective drape to the airway tube while the airway tube is inserted in the patient's mouth. For example, in some embodiments, as shown in FIG. 3 the airway tube 180 may be detached from an anesthesia circuit and an exposed proximal end of the airway tube 180 may be inserted through the aperture 150 in the protective drape 100. The airway tube 180 may then be attached to the anesthesia circuit, as shown in FIG. 2. Likewise, once the airway tube 180 is inserted through the aperture 150, the airway tube 180 may be secured to the protective drape 100 using the adhesive strip 160.

In some embodiments, the method may involve disconnecting the airway tube from the anesthesia circuit (for example, as shown in FIG. 3) before the airway tube is removed from the patient. In other embodiments, parts of the anesthesia circuit may remain connected to the airway tube during removal, which can help to contain any fluids that are present in the airway tube.

### IV. Conclusion

Examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to the skilled artisan.

It is understood that the invention is not limited to the particular methodology, protocols, etc., described herein, as these may vary as the skilled artisan will recognize. It is also to be understood that the terminology used herein is used for the purpose of describing particular embodiments only, and is not intended to limit the scope of the invention. It also is to be noted that, as used herein and in the appended claims, the singular forms “a,” “an,” and “the” include the plural reference unless the context clearly dictates otherwise. Thus, for example, a reference to “a structure” is a reference to one or more structures and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Any numerical values recited herein include all values from the lower value to the upper value in increments of one unit provided that there is a separation of at least two units between any lower value and any higher value. As an example, if it is stated that the concentration of a component or value of a process variable such as, for example, size and the like, is, for example, from 1 to 90, specifically from 20 to 80, more specifically from 30 to 70, it is intended that values such as 15 to 85, 22 to 68, 43 to 51, 30 to 32, etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Particular methods, devices, and materials are described, although any methods and materials similar or equivalent to those described herein can be used in the practice or testing of the invention. The disclosures of all references and publications cited above are expressly incorporated by reference in their entireties to the same extent as if each were incorporated by reference individually.

What is claimed is:

1. A protective drape for a medical procedure, the protective drape comprising:
a protective sheet including an interior surface, an exterior surface, a first end, a second end, a first side, and a second side; and
a first flap coupled to the interior surface of the protective sheet so as to form a pocket at the first end of the protective sheet, the first flap including:
an outer edge positioned away from the second end of the protective sheet and connected to the protective sheet, and
a first lateral edge that is connected to the protective sheet so as to form a sealed periphery of the pocket that extends over the outer edge and a first lateral edge of the first flap.

2. The protective drape of claim 1, wherein the first flap includes a second lateral edge that is also connected to the protective sheet so as to continue the sealed periphery of the pocket over the second lateral edge of the first flap.

3. The protective drape of claim 1, wherein the first flap extends across the protective sheet from the first side to the second side.

4. The protective drape of claim 1, wherein the first end of the protective sheet includes a first edge, and wherein the first edge of the protective sheet is aligned with the outer edge of the first flap.

5. The protective drape of claim 4, wherein the first flap and the protective sheet are formed by a single material sheet, and
wherein the first edge of the protective sheet and the outer edge of the first flap are connected by a fold in the single material sheet.

6. The protective drape of claim 1, wherein the first lateral edge of the first flap is connected to the protective sheet by a seam.

7. The protective drape of claim 1, further comprising a first wing extending laterally outward from the first side of the protective sheet.

8. The protective drape of claim 7, wherein the protective sheet and the first wing are formed by a single material sheet.

9. The protective drape of claim 1, further comprising an aperture extending through a central region of the protective sheet.

10. The protective drape of claim 9, further comprising a second flap coupled to the interior surface of the protective sheet so as to form a second pocket at the second end of the protective sheet, the second flap including:
an outer edge positioned away from the first end of the protective sheet and connected to the protective sheet, and
a first lateral edge that is connected to the protective sheet so as to form a sealed periphery of the pocket that extends over the outer edge and a first lateral edge of the second flap.

11. The protective drape of claim 9, further comprising an adhesive strip secured to the protective sheet adjacent to the aperture, the adhesive strip being configured to attach to a tube passing through the aperture.

12. The protective drape of claim 11, wherein the adhesive strip is attached to the exterior surface of the protective sheet.

13. The protective drape of claim 9, further comprising an adhesive strip secured to the protective sheet at the second end of the protective sheet, the adhesive strip being configured to attach to a tube passing adapted for use with the protective drape.

14. The protective drape of claim 1, wherein the protective sheet is transparent.

15. The protective drape of claim 1, wherein the protective sheet is formed of a polyfilm material.

16. A method comprising:
removing a distal end of an airway tube from a patient, wherein a proximal end of the airway tube is secured to a protective drape, wherein the protective drape comprises:
a protective sheet including an interior surface, an exterior surface, a first end, a second end, a first side, and a second side, and
a first flap coupled to the interior surface of the protective sheet so as to form a pocket at the first end of the protective sheet, the first flap including:
an outer edge positioned away from the second end of the protective sheet and connected to the protective sheet, and
a first lateral edge that is connected to the protective sheet so as to form a sealed periphery of the pocket that extends over the outer edge and a first lateral edge of the first flap; and
securing the distal end of the airway tube over the interior surface of the protective drape such that fluids from the airway tube drip onto the protective drape and run into the pocket.

17. The method of claim 16, wherein the first flap includes a second lateral edge that is also connected to the protective sheet so as to continue the sealed periphery of the pocket over the second lateral edge of the first flap.

18. The method of claim 16, wherein the first flap extends across the protective sheet from the first side to the second side.

19. The method of claim 16, wherein the first end of the protective sheet includes a first edge, and wherein the first edge of the protective sheet is aligned with the outer edge of the first flap.

20. The method of claim 16, wherein the first flap and the protective sheet are formed by a single material sheet, and wherein the first edge of the protective sheet and the outer edge of the first flap are connected by a fold in the single material sheet
